(19) United States
(12) Patent Application Publication
(10) Pub. No.: US 2018/0125582 A1
(43) Pub. Date: May 10, 2018

(54) CORNEAL SURGERY RISK EVALUATION METHOD AND SYSTEM THEREOF

(71) Applicant: Argusys, Inc., San Jose, CA (US)

(72) Inventors: Wen-Pin SHIH, San Jose, CA (US); Wen-Chin CHEN, San Jose, CA (US)

(21) Appl. No.: 15/807,230

(22) Filed: Nov. 8, 2017

(30) Foreign Application Priority Data
Nov. 8, 2016 (TW) 105136309

(51) Int. Cl. A61B 34/10; A61B 3/00; A61B 3/14; A61B 3/16; A61F 9/013

(52) U.S. Cl. CPC A61B 34/10; A61B 3/0025; A61B 3/14; A61B 2017/320052; A61F 9/013; A61B 2034/105; A61B 2034/104; A61B 3/16

(57) ABSTRACT

This invention provides a corneal surgery risk evaluation method and system thereof. Utilizing a mechanical numerical model to evaluate stress differences before corneal surgery and after, and further providing a suggested surgical path and risk after surgery. The evaluation method, comprising: measuring Intraocular pressure (IOP); inputting geometric parameters and material parameters of multi-layer of corneal; constructing a first corneal numerical model; constructing a second corneal numerical model with at least one cutting path character; evaluating whether the cutting path character should be re-constructed or not.

### BACKGROUND OF THE INVENTION

### 1. Field of the Invention

The present invention relates to a mechanic evaluation method and system, especially a corneal surgery risk evaluation method and system thereof after calculating by a numerical model.

### 2. Description of the Prior Art

In general, the process of normal human eye to receive light source and generate an image is that the refractive light source by cornea passes through pupil, and then adjusting the intensity of the light source by pupil dilation or miosis actuated by iris. Then the light source refracts via lens and focuses on the retina to form the image. The image is transmitted to brain through optic nerve. In said process, the spherical curvature of cornea is very important. If the function is normal, the light source can be refracted correctly and focused clearly on the retina. If the surface of the retina is abnormal or the thickness is uneven, the image will not be focused on the retina so that a blurred vision will occur.

If the spherical curvature of the cornea of the eye is greater than the normal eye spherical curvature of the cornea. When the light enters into the eye, it is focused in front of the retina so that the far image is not clear, this phenomenon is myopia. Conversely, if the spherical curvature of the cornea of the eye is smaller than the normal eye spherical curvature of the cornea. When the light enters into the eye, it is focused behind the retina so that the near image is not clear, this phenomenon is hyperopia. In addition, if the corneal spherical curvature is uneven resulting in an image which is not concentrated will form astigmatism phenomenon.

In addition to the above-mentioned problems, there are corneal lesions, ulcers or injury resulting in irregular corneal spherical curvature which leads to blurred vision. In severe cases, the cornea must be replaced.

The disease caused by abnormal concentration of eyes mentioned above generally referred to refractive errors. In addition to the conventional correction method (i.e., wearing glasses or contact lenses in general) to improve the focus function, currently there is the Ray Radiation surgery correction.

Currently the common methods are Radial Keratotomy (RK), Photorefractive Keratectomy (RRK), Laser in Situ Keratomileusis (LASIK), and Small Incision Lenticule Extraction (SMILE).

Corneal replace surgery is first to remove the damaged cornea to make the lens appear. Then the cornea from donor will be sutured to the cornea and sclera of the patient by the doctor, and a radial suture line will remain.

However, before current surgical operation, there is no more accurate evaluating means for doctors to consult. It depends on a rule of thumb, empirical formula and physician experiences. In the surgical procedure, there are also other factors (such as medicine physician wield special eye mechanical properties of the cornea is not completely grasp) may cause failures of surgery, ineffective operation, or mild impact causes rupture and sequela. For example, sight reply (deterioration) in postoperative, conical cornea, corneal rupture after suture causes sight cannot be restored, and so on. For those who need to surgery, it is insecure and uncertain.

### SUMMARY OF THE INVENTION

One aspect of the present invention is to provide a corneal surgery risk evaluation method. Using a mechanical model to evaluate the stress difference before and after surgery, and to provide proposals of surgical path and postoperative risk. The evaluation method comprises the following steps: (S1) measuring an intraocular pressure (IOP); (S2) inputting geometric parameters and material parameters of multi-layer of cornea; (S3) constructing a first corneal numerical model; (S4) constructing a second corneal numerical model with at least one cutting path character; and (S5) evaluating whether the cutting path character should be re-constructed or not. Wherein the geometrical parameters and material parameters are extracted out during measuring the intraocular pressure.

Another aspect of the present invention is to provide a corneal surgery risk evaluation system. Evaluating the stress difference before and after the surgery through the evaluation method built in the system, and providing proposals of surgical path and postoperative risk. The system comprises a tonometer for providing an external force to cornea and measuring an intraocular pressure; a camera for measuring a deformation behavior of the cornea; and a processor connected to the tonometer and the camera. The evaluation method is built/stored in the processor.

Another aspect of the present invention is to provide a corneal surgery risk evaluation method. Using a mechanical model to evaluate the stress difference before and after surgery, and to provide proposals of surgical path and postoperative risk. The evaluation method comprises the following steps: (A1) measuring an intraocular pressure (IOP); (A2) inputting geometric parameters and material parameters of multi-layer of cornea; (A3) constructing a first corneal numerical model, wherein, using a yield strength of each of the multi-layer as a standard, defining dangerous area if it exceeds the yield strength, defining warning area if it is 60˜100% of the yield strength, and defining safe area if less than 60% of the yield strength; (A4) constructing a second corneal numerical model having at least one cutting path character; (A5-1) inputting a normal IOP value into the second corneal numerical model for simulating, and comparing to the first corneal numerical model, configured to evaluate whether the dangerous area exceeds 5% of whole area of the cornea, or whether the warning area exceeds 20% of whole area of eye; (A5-2) inputting an abnormal IOP value into the second corneal numerical model for simulating, and comparing to the first corneal numerical model, configured to evaluate whether the dangerous area exceeds 10% of whole area of the cornea, or whether the warning area exceeds 50% of whole area of eye; (A5-3) inputting a rubbing-eye IOP value and an external force of a tangent direction or a torque of the cornea into the second corneal numerical model for simulating, and comparing to the first corneal numerical model, configured to evaluate whether the dangerous area exceeds 20% of whole area of the cornea, or whether the warning area exceeds 60% of whole area of eye; (A5-4) inputting a rubbing-eye IOP value and an external force of a tangent direction or a torque of the cornea into the second corneal numerical model for simulating, and comparing to the first corneal numerical model, configured to evaluate whether a stress in any area of each of the multi-layer exceeds 5 times the normal IOP value; and (A5-5) inputting a normal IOP value into the second corneal numerical model for simulating, and comparing to the first corneal numerical model, configured to evaluate whether an arc stress in any area of each of the multi-layer exceeds 15%. Wherein the geometrical parameters and material parameters are extracted out during measuring the intraocular pressure.

Compared to the prior art, the present invention can evaluate difference before and after surgery through a mechanical model and provide a proposed surgical way. It can provide a safer and more accurate evaluation way.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram of a multi-layered structure of a cornea.

FIG. 1B is a schematic diagram of material parameters of the cornea.

FIG. 2 is a flowchart of one embodiment of the present invention.

FIG. 3 is a stress-strain graph diagram of the cornea.

FIG. 4A-4D are mechanical distribution diagrams of RK, PRK, LASIK, and SMILE surgeries.

FIG. 4E-4H are schematic diagrams of postoperative cornea deformation.

FIG. 5A-1-5A-4, 5B-1, 5B-2, 5C-1, 5C-2, 5D-1, and 5D-2 are cutting path characters after RK, PRK, LASIK, SMILE surgeries.

FIG. 6A-1, 6A-2, 6B-1, 6B-2, 6C-1, 6C-2, 6D-1, 6D-2, 6E-1, 6E-2, 6F-1, 6F-2, 6G-1, 6G-2, 6H1, and 6H-2 are corneal cracking potential distributions after RK, PRK, LASIK, and SMILE surgeries.

FIG. 7 is a schematic diagram of an evaluation system of the present invention.

FIG. 8A and 8B are flowcharts of another embodiment of the present invention.

### DETAILED DESCRIPTION OF THE PREFERRED EMBODIMENT

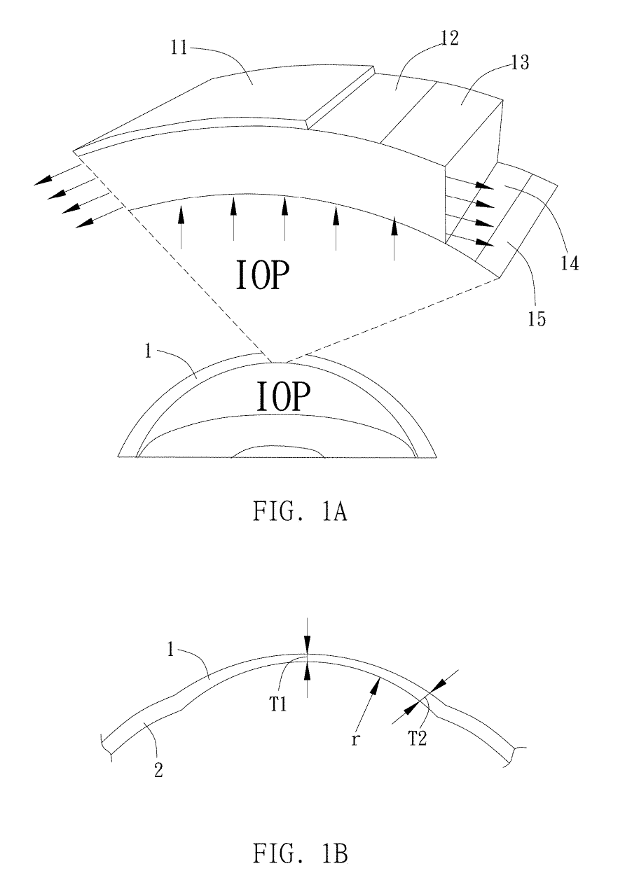

Please refer to FIG. 1A and FIG. 1B. FIG. 1A is a schematic diagram of a multi-layered structure of a cornea 1. The cornea 1 comprises Epithelial Layer 11, Anterior Elastic Lamina/Bowman's Membrane 12, Stroma 13, Posterior Elastic Lamina/Descemet's Membrane 14, and Endothelium Layer 15. In this embodiment, it is mainly to discuss Anterior Elastic Lamina/Bowman's Membrane 12, Stroma 13, and Posterior Elastic Lamina/Descemet's Membrane 14. FIG. 1B a schematic diagram of material parameters of the cornea 1. The material parameters at least comprise radius r, thickness T1, and thickness T2. Wherein the thickness T1 is preferably refer to location near the center of the cornea. The thickness T2 is preferably refer to location near the end of the cornea (i.e. location much closer to sclera 2).

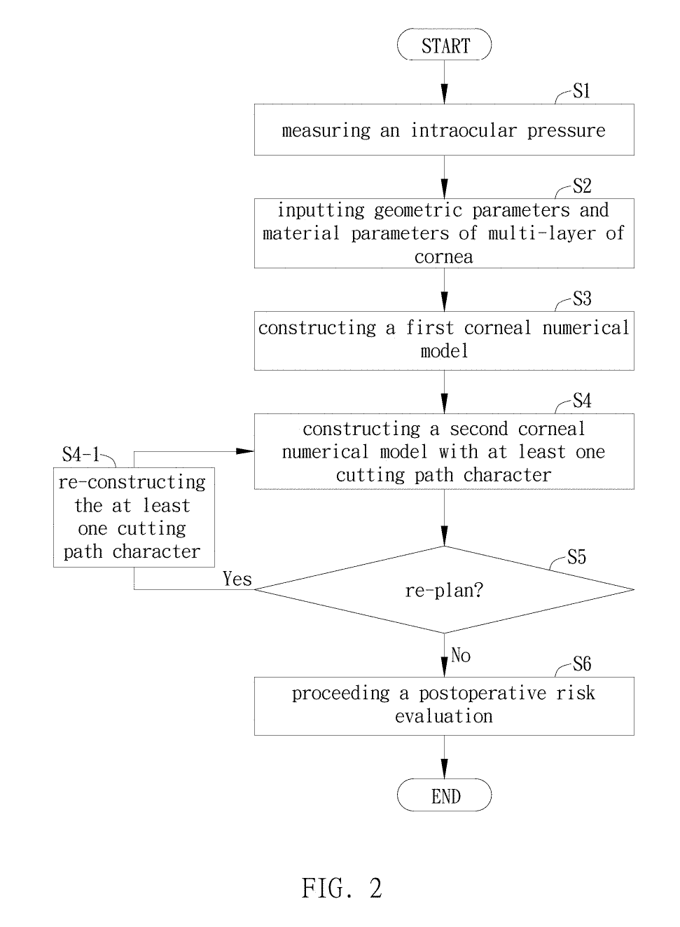

Please refer to FIG. 2, corneal surgery risk evaluation method in the present embodiment preferably comprises the following steps: (S1) measuring an intraocular pressure (IOP); (S2) inputting geometric parameters and material parameters of multi-layer of cornea; (S3) constructing a first corneal numerical model; (S4) constructing a second corneal numerical model with at least one cutting path character; and (S5) evaluating whether the cutting path character should be re-constructed or not. Wherein the geometrical parameters and material parameters are extracted out during measuring the intraocular pressure.

Step (S1) measuring an intraocular pressure (IOP). Using a tonometer to provide an external force to cornea and measure an intraocular pressure. In this embodiment, the tonometer can be a Pneumatonometer, a contact or non-contact tonometer, but not limited thereto.

Step (S2) inputting geometric parameters and material parameters of multi-layer of cornea. For example, analyzing a corneal disturbance and the geometric parameters results from an external force from the tonometer through an image processing of a camera. Further to use the image processing of the camera to obtain the multi-layer of cornea. Accordingly, we can analyze the geometric parameters and the material parameters of multi-layer of cornea. The geometric parameters comprise the curvature R and thickness T distribution of whole cornea. The curvature R distribution can be converted by radius r. The thickness T distribution comprises T1 and T2 mentioned above.

It should be noted that the extracted way of the above-mentioned geometric parameters and the material parameters of multi-layer of cornea can refer to Po-Jen Shih, Huei-Jyun Cao, Chun-Ju Huang, I-Jong Wang, Wen-Pin Shih and Jia-Yush Yen, “A corneal elastic dynamic model derived from Scheimpflug imaging technology ”, Ophthalmic Physiol Opt 2015, 35, 663-672. This reference is incorporated by reference herein.

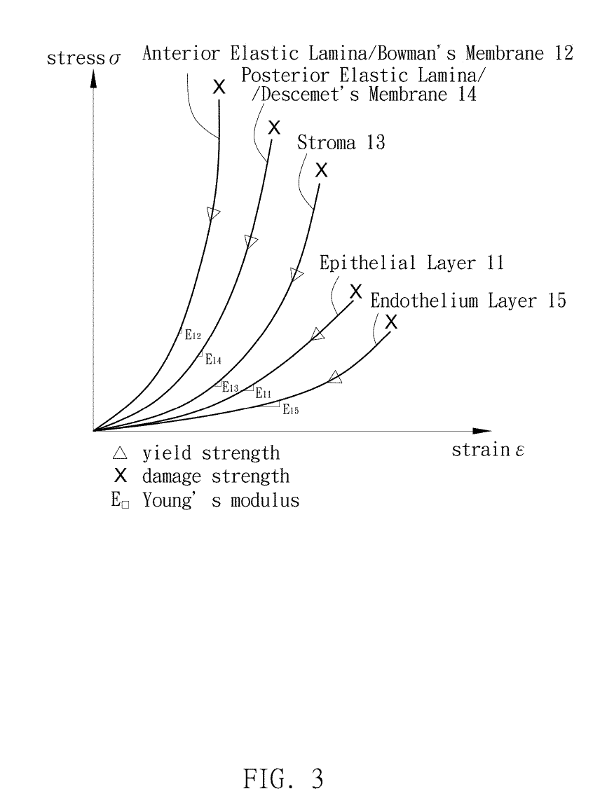

The material parameters of the cornea obtained through image processing of the camera include but not limited to Young's modulus, Poisson ratio, yield strength and breaking strength in each layer, as shown in FIG. 3. It is noted that in this embodiment, it is mainly analyzed/obtained each curvature R, thickness T, Young's modulus, Poisson ratio, yield strength and breaking strength of whole cornea of Anterior Elastic Lamina/Bowman's Membrane12, Stroma13 and Posterior Elastic Lamina/Descemet's Membrane14. In other embodiments, the Epithelial Layer and Endothelium Layer can also be analyzed.

The obtained geometric parameters and the material parameters are inputted into a numerical analysis software/model, e.g. ANSYS, but not limited thereto.

Step (S3) constructing a first corneal numerical model. That is, by the above-mentioned geometrical parameters and material parameters, and to establish a first corneal numerical model through a numerical analysis software/model. The model is built based on finite element analysis, but not limited thereto. The model from this analysis is in accordance with layered model of the cornea.

Step (S4) constructing a second corneal numerical model with at least one cutting path character. Similarly, establishing a second corneal numerical model through a numerical analysis software/model. For example, according to the first corneal numerical model, further to modify geometrical configuration and boundary conditions. The model should be selected from suitable elements for analyzing three-dimensional layered structure. In addition, according to different cutting path character for different operations (e.g., RK, PRK, LASIK, SMILE surgeries or other corneal surgery), such as cutting range, cutting pattern, cutting length, cutting depth, which is built into the second numerical model, to form a free boundary condition of partial surface region. In the first time to establish the second corneal numerical model, it can be modeled by the existing rule of thumb or formula. Numerical simulation analysis results show before and after surgery, the stress and strain distribution of the cornea.

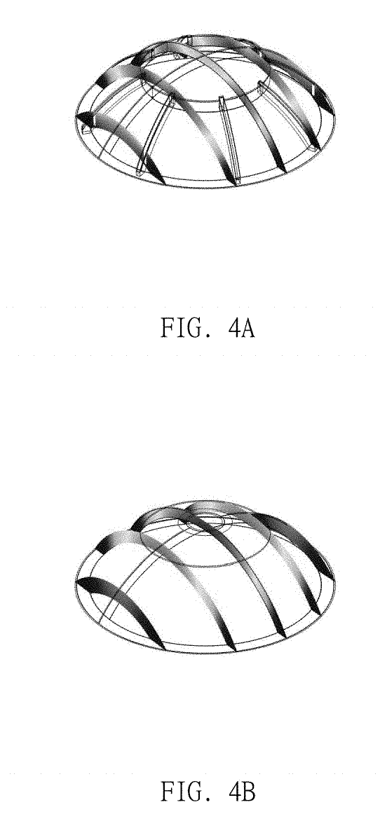

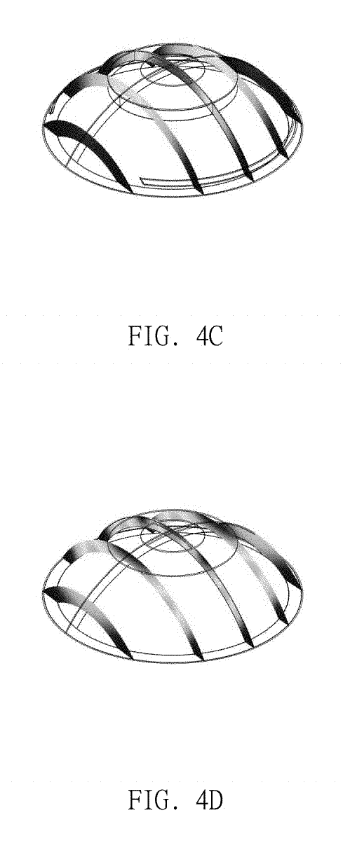

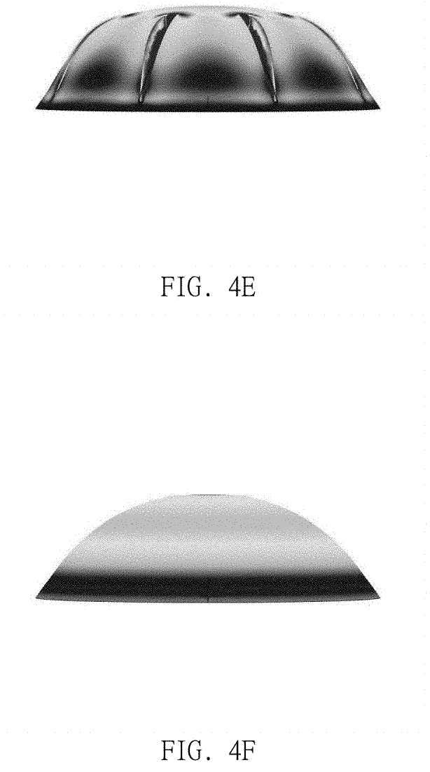

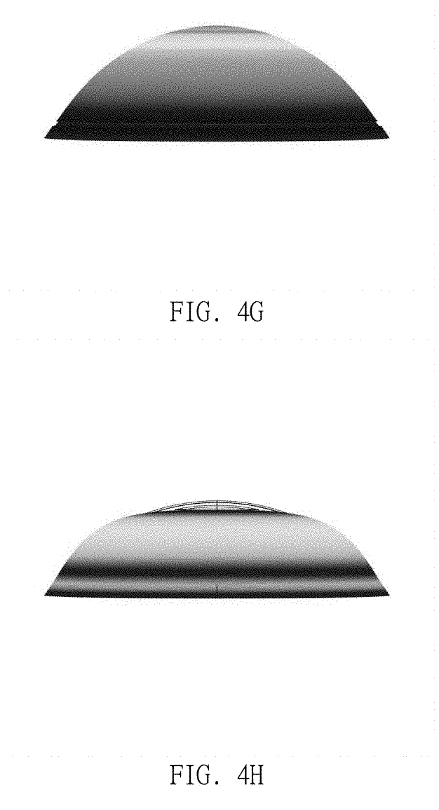

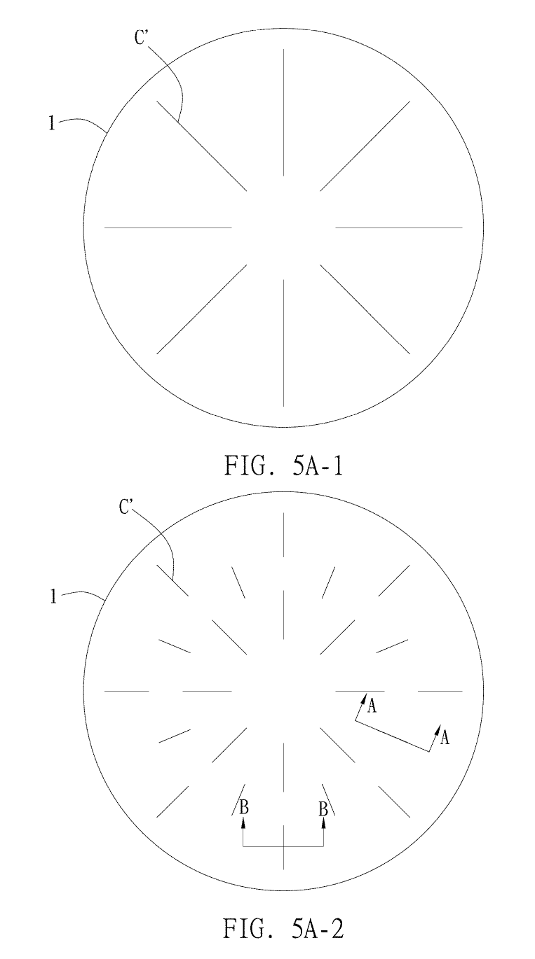

It is noted that, in this embodiment, the stress analysis mainly considers three-layer structure of the layered cornea, i.e., Anterior Elastic Lamina/Bowman's Membrane12, Stroma13 and Posterior Elastic Lamina/Descemet's Membrane14, but not limited thereto. The stress of the three layers is preferably according to the yield strength of each layer as a standard. It is defined that if it exceeds the yield strength, the region is marked in red (i.e. danger), the red (danger) region may be regarded as a force relatively large area. If it is 60%-100% of the yield strength, the region is marked in orange/yellow (warning). If less than 60% of the yield strength are marked in green (safety). The color under green (including blue and indigo) could also be regarded as safety, as shown in FIG. 4A-FIG. 4D, the sequence diagram respectively represents the surgical mechanical distribution of RK, PRK, LASIK, and SMILE. Wherein the yield strength is according to a tensile test of each layer, the stress value of a gently point (Δ may be designated at the reference to FIG. 3) of a slope from an experiment. However, in different embodiments, the observing standard may be different stress/strain mechanical parameters or other values.

Step (S5) evaluating whether the cutting path character should be re-constructed or not. In this step, using the yield strength as the evaluation basis. If the evaluation result is unsafe, for example, the model displays red or orange/yellow areas exceed a certain distribution area, then proceeding step (S4-1) re-constructing the at least one cutting path character, and executing the step (S4) again. That is, create a new second corneal numerical model having at least one cutting path character.

Evaluating whether the cutting path character should be re-constructed or not can proceed in five aspects. For example, in one embodiment, inputting a normal IOP value (e.g. 10-20 mmHg) into the second corneal numerical model for simulating, and comparing to the first corneal numerical model, configured to evaluate whether the dangerous area exceeds 5% of whole area of the cornea, or whether the warning area exceeds 20% of whole area of eye, or the red or orange/yellow areas increase a certain percentage compared to the first corneal numerical model, it must be re-planning a second corneal numerical model with another cutting path character.

In another embodiment, inputting an abnormal IOP value (e.g. 25-35 mmHg) into the second corneal numerical model for simulating, and comparing to the first corneal numerical model, configured to evaluate whether the dangerous area exceeds 10% of whole area of the cornea, or whether the warning area exceeds 50% of whole area of eye, or the red or orange/yellow areas increase a certain percentage compared to the first corneal numerical model, it must be re-planning a second corneal numerical model with another cutting path character.

In another embodiment, inputting a rubbing-eye IOP value (e.g. 40-60 mmHg) and an external force (e.g. 0.5N) of a tangent direction or a torque (e.g. 0.5 N-cm) of the cornea into the second corneal numerical model for simulating, and comparing to the first corneal numerical model, configured to evaluate whether the dangerous area exceeds 20% of whole area of the cornea, or whether the warning area exceeds 60% of whole area of eye, or the red or orange/yellow areas increase a certain percentage compared to the first corneal numerical model, it must be re-planning a second corneal numerical model with another cutting path character.

The tangent direction refers to apply to a circular area about 0.25 cm radius of the central cornea in a horizontal direction. The torque is a force clockwise or counterclockwise which is applied to circular area about 0.25 cm radius of the central cornea.

In another embodiment, inputting a rubbing-eye IOP value (e.g. 40-60 mmHg) and an external force (e.g. 0.5N) of a tangent direction or a torque (e.g. 0.5 N-cm) of the cornea into the second corneal numerical model for simulating, and comparing to the first corneal numerical model, configured to evaluate whether a stress in any area of each of the multi-layer exceeds 5 times the normal IOP value. If the stress in any area of Anterior Elastic Lamina/Bowman's Membrane12, Stroma13 and Posterior Elastic Lamina/Descemet's Membrane14 exceed 5 times the normal IOP value, it must be re-planning a second corneal numerical model with another cutting path character. As shown in FIG. 4E˜FIG. 4H, FIG. 4E indicates a deformation after RK, FIG. 4F indicates a deformation after PRK, FIG. 4G and FIG. 4H indicate deformations after LASIK and SMILE respectively.

In another embodiment, inputting a normal IOP value (e.g. 10-20 mmHg) into the second corneal numerical model for simulating, and comparing to the first corneal numerical model. If it is evaluated that the arc stress in any area of each layer of Anterior Elastic Lamina/Bowman's Membrane12, Stroma13 and Posterior Elastic Lamina/Descemet's Membrane14 exceeds 15%, it must be re-planning a second corneal numerical model with another cutting path character.

The cutting path character is preferred to reduce the stress concentration area range (i.e. red region area) and magnitude as a principle. Then adjusting the change in strain uniformly, especially in the region of the optical center of the cornea. The cutting path character further comprises modifying the problem of stress concentration of the conventional technology.

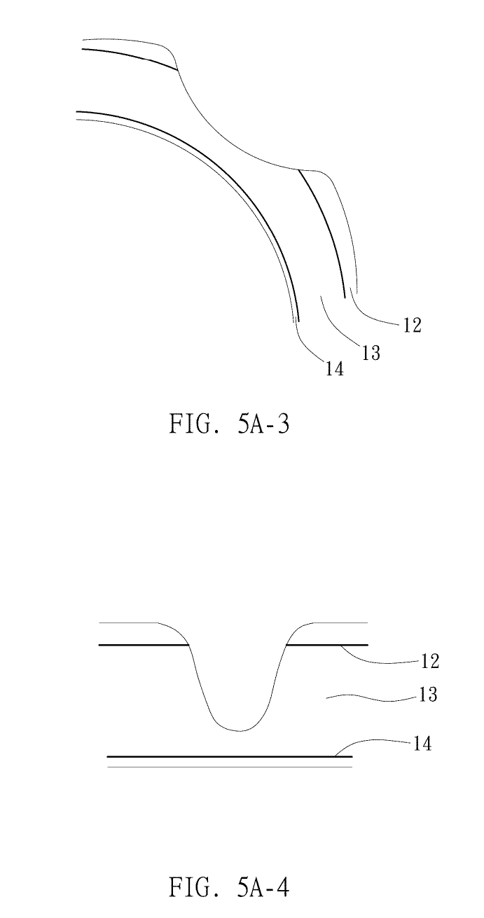

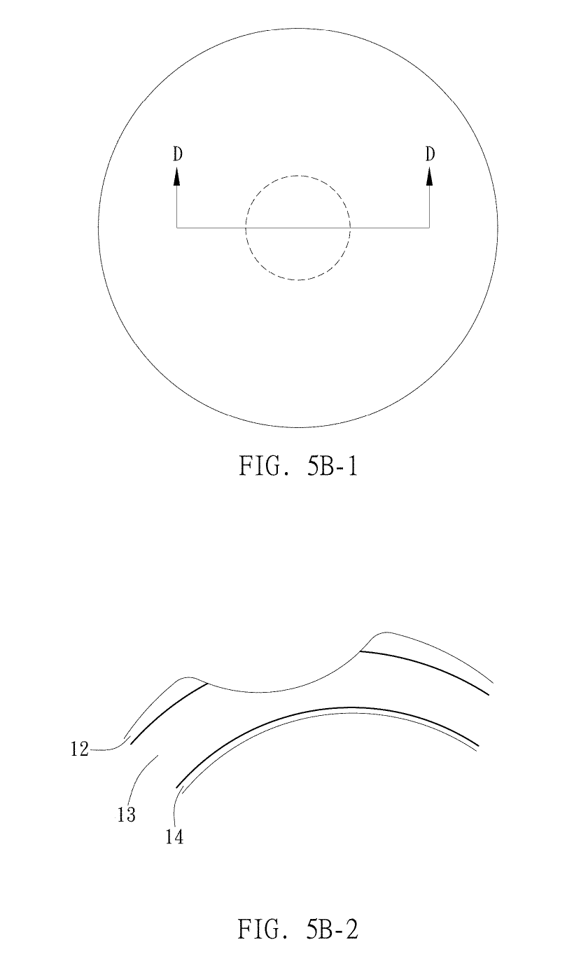

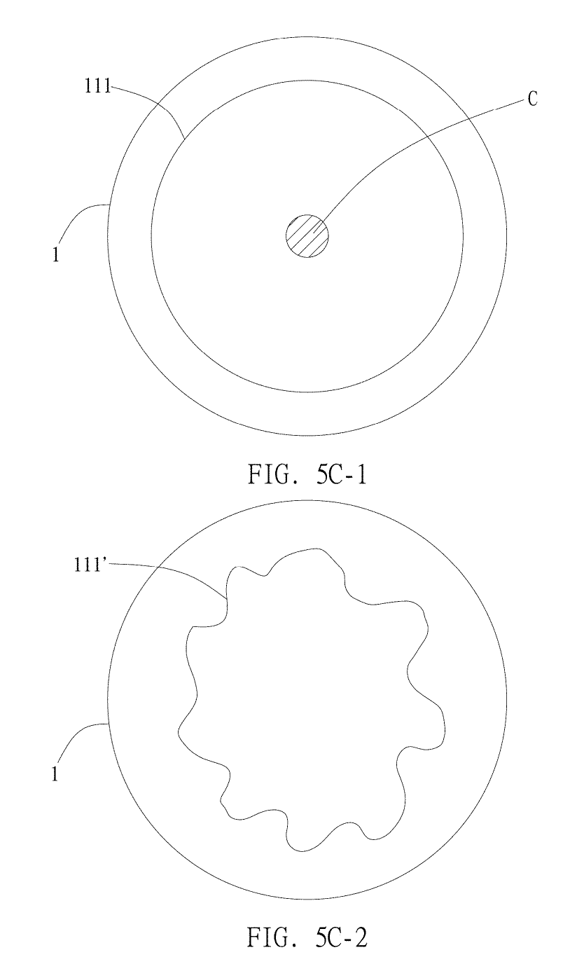

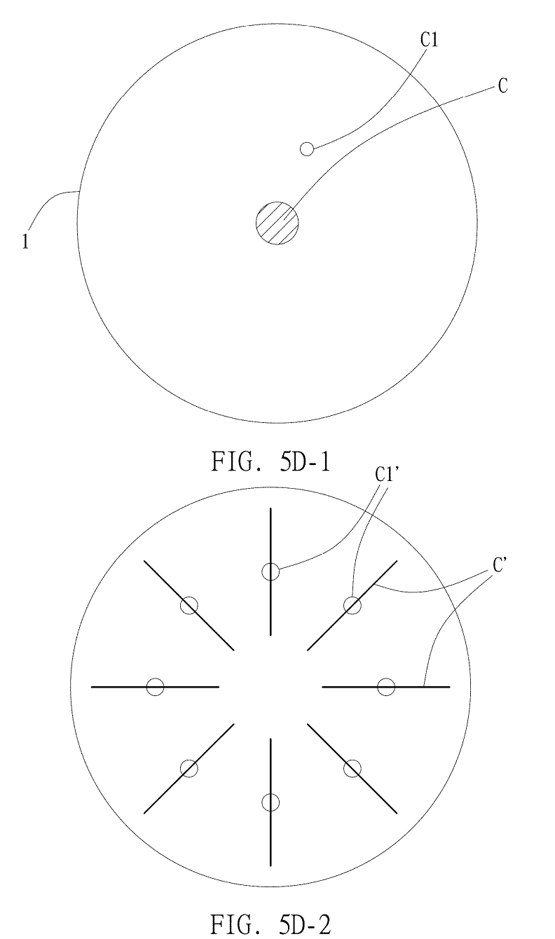

Please refer to FIG. 5A-1˜FIG. 5A-4. The cutting path characters are evaluated from the numerical models applied to RK, PRK, LASIK, SMILE, respectively.

As shown in FIG. 5A-1˜FIG. 5A-4, in this embodiment, it takes RK for example. FIG. 5A-1 shows original RK surgical pattern C. After evaluation by the model, it is found to need to re-plan. FIG. 5A-2 shows a new re-planned cutting path character after evaluation. FIG. 5A-3 and FIG. 5A-4 represent the section view of C′ from A-A and B-B of FIG. 5A-1 and FIG. 5A-2, respectively. In order to avoid the stress value of the cutting line end corner is too large, it is re-planned to change the radial cutting end corner to become a parabolic curve notch. Moreover, decreasing the cutting length of the radial line and increasing the number of radial line and arranged in an interlaced manner. It can reduce the stress concentration region and reduce the largest stress value thereof. It reduces the strain and makes the change uniformly.

As shown in FIG. 5B-1˜FIG. 5B-2. We take a central region cutting manner of PRK surgery for example. With respect to the patient who needs to a larger correction prescription, cutting and shaping the central area of cornea directly needs a larger circular area for cutting. However, the larger circular area is cut, the larger area of Anterior Elastic Lamina/Bowman's Membrane12 is damaged. This could make the intensity of the upper cornea be insufficient. In addition, because the IOP exists, the cut area may bulge toward outside, so that the effect to the myopia patient may be negative. Accordingly, the improved cutting path character may have smaller cutting area (i.e. smaller damaged area of Anterior Elastic Lamina/Bowman's Membrane12) according to patient's geometrical radius of eye, IOP status, and corneal thickness distribution chart which are analyzed with mechanical deformation and optics. As shown in FIG. 5B-2, with respect to the central cutting area, the cutting surface is replaced to a notched paraboloid.

As shown in FIG. 5C-1˜FIG. 5C-2, the present embodiment takes a cutting method of central area of LASIK surgery and the corneal flap 111 as an example. Traditional LASIK surgery (see FIG. 5C-1) must firstly open the corneal flap 111, and then perform a cutting action with cutting surface C on the central optical area. The strain transmission of the corneal flap 111 and the lower cornea may be not continuous due to the corneal flap 111 is cut, opened, and restored. This may cause the hoop stress of the corneal flap 111 is insufficient, so that it easily separates after compressing. In order to increase the joint force between the corneal flap 111 and the original cornea 1, the traditional circular curve 111 is replaced by the re-planned cutting path geometrical pattern formed a petal shape 111′ (see FIG. 5C-2).

As shown in FIG. 5D-1˜FIG. 5D2, this embodiment takes the SMILE surgery as an example. Traditional SMILE cuts the central area of the cornea through a minimally invasive incision C1 to shape. However, in the minimally invasive technique, it is not easy to clean up the cutting area C (inside the cornea 13), so that it is not stable. The new cutting path character from the numerical model is based on the minimally invasive technique to damage small part C1′ (i.e. minimally invasive incision) of the Anterior Elastic Lamina/Bowman's Membrane12 further with the radial cutting path C′ (inside the cornea 13) of RK, so that the central optical area will not be damaged. Accordingly, the purpose of refractive correction.

The above embodiment is for needing to re-plan. When the evaluation is not to re-plan, then proceeding step (S6) proceeding a postoperative risk evaluation. The postoperative risk evaluation in this embodiment is for the healthcare professional to inform the patient according to the above simulation and evaluation result in advance that the eyes may have variations after surgery or the notification in future life. Accordingly, the patient could choose whether to accept such surgery, and the willingness to bear the risk of surgery.

In addition, when one week after the patient goes under the surgery, further performing step (S7) according to step (S1)˜step (S2): constructing a third corneal numerical model. To input an abnormal IOP (e.g. 25˜35 mmHg) and simulate rubbing-eye status (e.g. setting IOP is 40˜60 mmHg, external force (e.g. 0.5N) of a tangent direction and torque (e.g. 0.5 N-cm)) for finding out the high stress potential concentration area. Further to perform step (S8): establishing a postoperative patient safety recommendation. The safety recommendation comprises a variety of life movements, sports and environmental restrictions.

The postoperative risk evaluation and the postoperative patient safety recommendation are divided postoperative situation simulation and postoperative notice. The postoperative situation simulation, in practice, regulating IOP value: notice patient regulating IOP value in present surgery. That is, using the simulation of the numerical model and the evaluation result to notice patient regulating the accurate IOP (e.g. regulate +5 mmHg). The significance of its application is that after surgery, the cornea will become thinner, if the normal intraocular pressure measurement of intraocular pressure, the intraocular pressure will be underestimated. Therefore, for patients with potential high intraocular pressure or glaucoma, unadjusted intraocular pressure will allow potential patients to miss the best treatment time.

Secondly, e.g. glare Evaluation: Since the cornea by cutting after which the stress distribution is not uniform, the strain distribution will be uneven on the surface, such that the inner layer of the cornea reflected glare generated increases. The mating geometric distortion of the optical analysis results may be presented glare state operation, this effect may inform the advantage that the life of the patient when glare light is generated in advance.

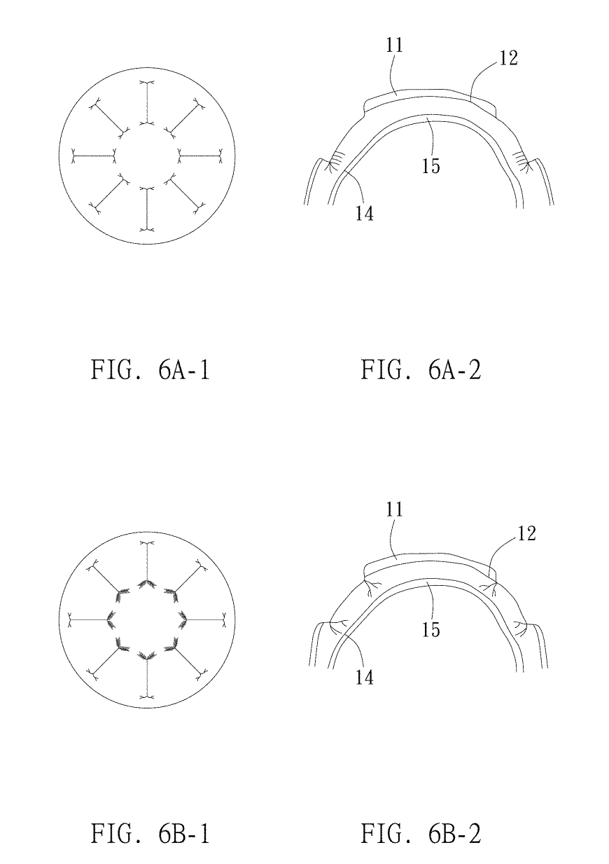

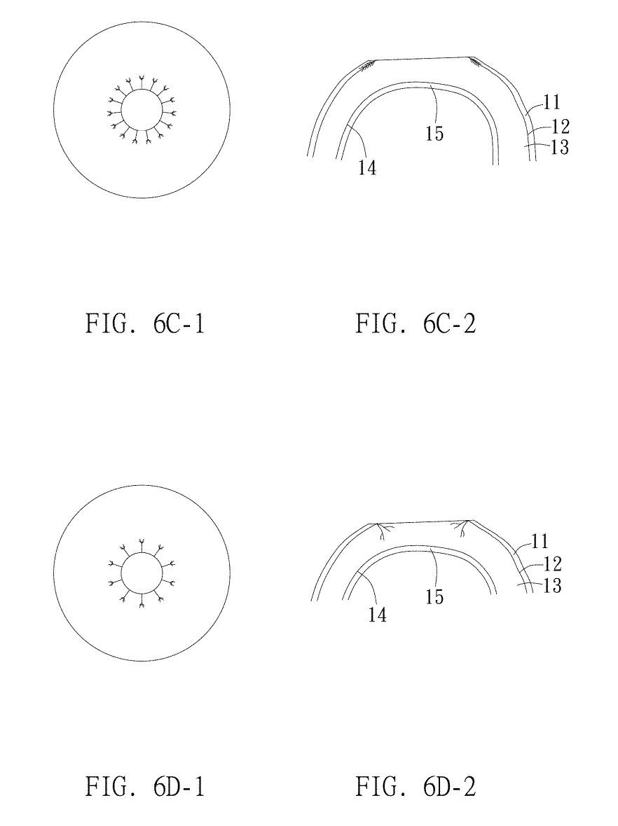

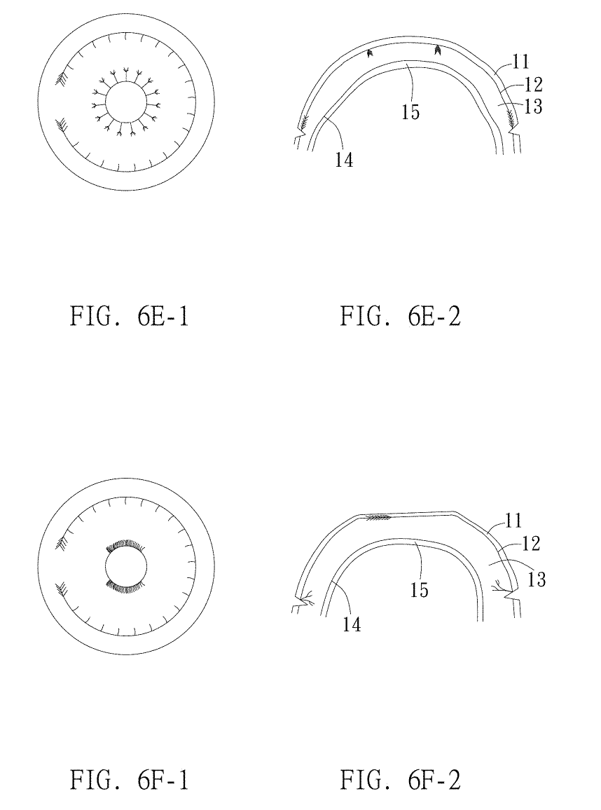

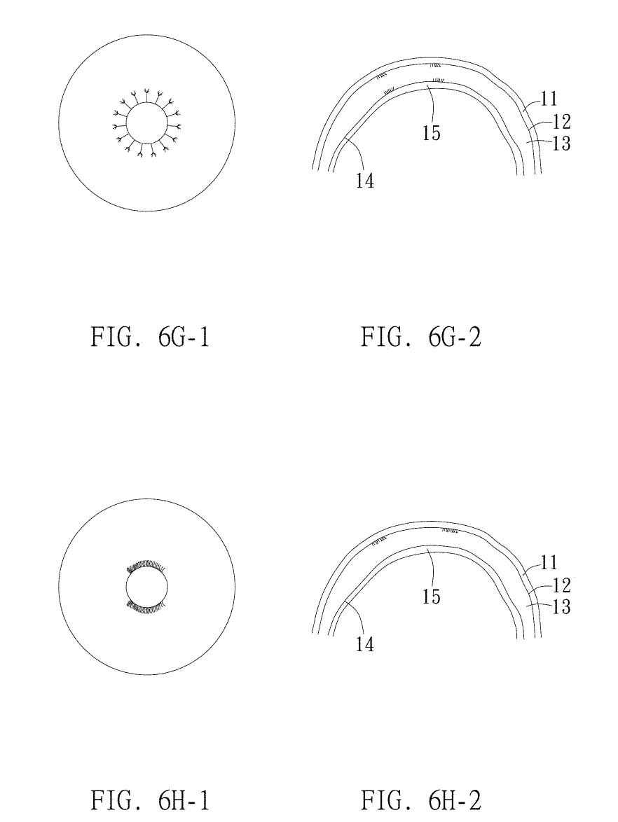

In addition, corneal cracking potential: when the cornea is cutting, the stress may be larger in partial area. With the material of the cornea, under the influence of external forces or human forces, the partial area may have a cracking potential. As shown in FIG. 6A-1˜FIG. 6H-2. FIG. 6A-1 and FIG. 6A-2 are schematic diagrams of corneal cracking potential distributions after RK. The cracking potential area is shown as tree structure. It is noted from the figures that the end (near the outside of the cornea, located on the lower cutting layer and near Posterior Elastic Lamina/Descemet's Membrane14) of the cutting path is easier to generate cracking.

As shown in FIG. 6B-1 and FIG. 6B-2, this embodiment is the potential cracking diagram which the cornea is rubbed after RK surgery. The cracking potential area is shown as tree structure. It is noted from the figures that the head end (near the inside of the central optical area of the cornea, located on the lower cutting layer and near Posterior Elastic Lamina/Descemet's Membrane14) of the cutting path is easier to generate cracking.

As shown in FIG. 6C-1 and FIG. 6C-2, this embodiment is the potential cracking diagram which the cornea is stressed after PRK surgery. The cracking potential area is shown as tree structure. It is noted from the figures that the edge of the cutting area is easier to generate cracking along the Anterior Elastic Lamina/Bowman's Membrane12.

As shown in FIG. 6D-1 and FIG. 6D-2, this embodiment is the potential cracking diagram which the cornea is rubbed after PRK surgery. The cracking potential area is shown as a tree branched structure. It is noted from the figures that the edge of the cutting area is easier to generate cracking downward.

As shown in FIG. 6E-1 and FIG. 6E-2, this embodiment is the potential cracking diagram which the cornea is stressed after LASIK surgery. The cracking potential area is shown as tree the branched structure. It is noted from the figures that the edge (a boundary of the breaking and the non-breaking cornea flap) of the lifted corneal flap and the inside edge of the cutting area are easier to generate cracking.

As shown in FIG. 6F-1 and FIG. 6F-2, this embodiment is the potential cracking diagram which the cornea is rubbed after LASIK surgery. The cracking potential area is shown as tree structure. Similarly, the edge (a boundary of the breaking and the non-breaking cornea flap) of the lifted corneal flap and the inside edge of the cutting area are easier to generate cracking.

As shown in FIG. 6G-1 and FIG. 6G-2, this embodiment is the potential cracking diagram which the cornea is stressed after SMILE surgery. In this case, the cutting boundary inside the cornea is easier to generate cracking.

As shown in FIG. 6H-1 and FIG. 6H-2, this embodiment is the potential cracking diagram which the cornea is rubbed after SMILE surgery. Similarly, the cutting boundary inside the cornea is easier to generate cracking.

The postoperative notice, in practice, likes to calculate the largest acceleration limitation which the cornea can sustain. The application is mainly for pilot or special occupation patient. The numerical model can provide the largest acceleration limitation which the cornea can sustain. The numerical model can calculate the largest acceleration limitation according to the acceleration of the cornea and analyze the stress distribution with the above 5 evaluated ways of the cutting path character.

Moreover, the rubbing-eye limitation: for the patient who likes rubbing eyes, the present embodiment provides the largest limitation of the maximum shear force which the cornea can withstand in postoperative. The numerical model can calculate the largest limitation of the maximum shear force according to the external force applied to the cornea and analyze the stress distribution with the above 5 evaluated ways of the cutting path character.

Further, the environmental pressure limitation: for the patient likes a diver or works at high pressure environment, the corneal numerical model provides the largest limitation of the maximum outer eye pressure which the cornea can sustain after surgery. The numerical model can calculate the largest pressure limitation according to the external pressure applied to the cornea and analyze the stress distribution with the above 5 evaluated ways of the cutting path character.

According to the above step (S1)˜step (S6), through the numerical model to evaluate the mechanical differences before and after surgery, and to provide recommendations of the surgical approach and a more secure and precise surgical evaluation methods.

When the postoperative risk evaluation is completed, the doctor informs patient that the probable risk or change after the corneal surgery. By the consent of the patient, the doctor can perform the corneal surgery.

According to the above step (S7)˜step (S8), the doctor notices the patient the safety precautions and restrictions of life after the corneal surgery through the patient safety recommendation.

In other embodiments, the evaluation method built from the numerical model, further to evaluate the corrected diopter, correct the corrected value from traditional IOP method after corneal surgery, evaluate the variation of the intensity of the corneal material after corneal surgery, evaluate the strength of corneal rupture caused by external force after corneal surgery, evaluate the potential corneal cracking risk after corneal surgery again after corneal surgery.

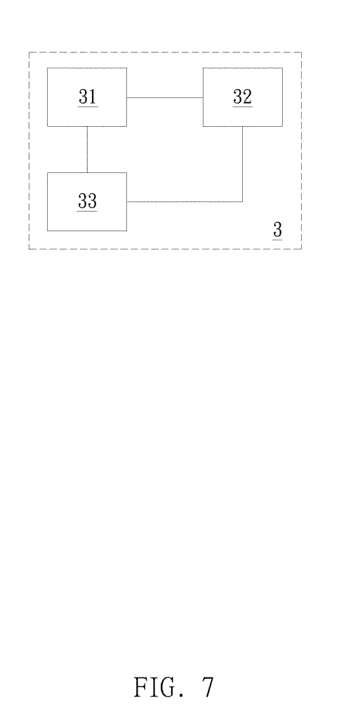

Another aspect of the present invention provides a corneal surgery evaluation system 3, as shown in FIG. 7. The evaluation system 3 preferably comprises a tonometer 31, a camera 32 and a processing system 33. The tonometer 31 is configured to provide an external force to the cornea 1 and measure the intraocular pressure. The camera 32 is configured to measure the corneal deformation behavior for the cornea 1. If necessary, it can photograph assisted with an added light source to analyze the geometrical parameters of each layer when the cornea 1 occurs dynamic disturbance from the external force, and further to obtain the material parameters of each layer of the cornea through the image processing of the camera 32. The processing device 33 connects to the tonometer 31 and the camera 32. The processing device 33 may be a computer, a smartphone, a tablet PC and the like.

It should be noted that the designer can write the above evaluation method into the processing device 33 by a firmware or software manner. In addition, a user interface can be designed in the processing device 33 to show each reference (data) from the evaluation method immediately.

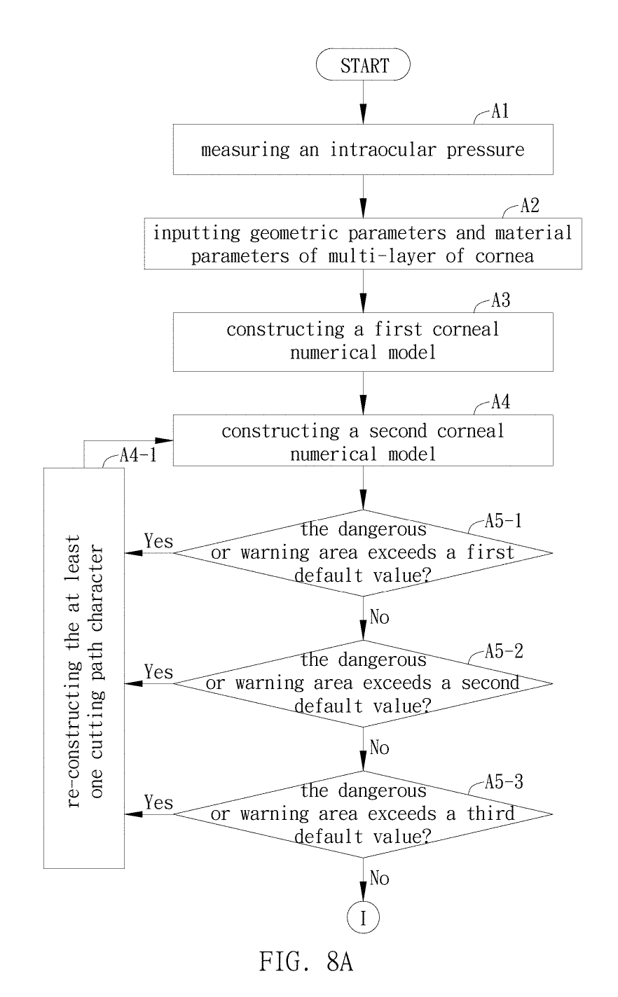

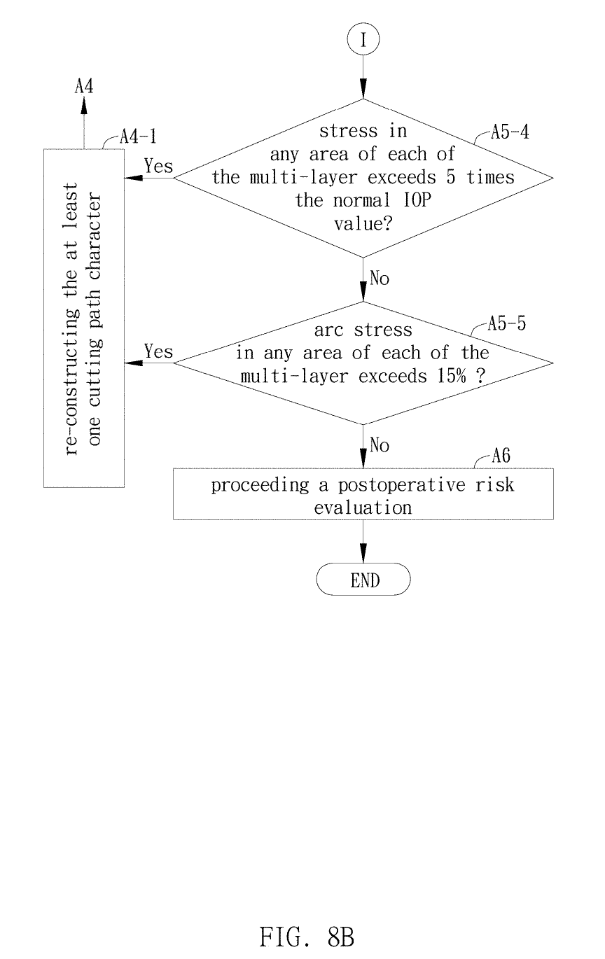

Another aspect of the present invention provides a corneal surgery risk evaluation method, through the numerical model to evaluate the mechanical stress difference before and after surgery, and provides a recommended surgical cutting path and postoperative risk. As shown in FIG. 8A˜FIG. 8B, the evaluation method preferably comprises the following steps: (A1) measuring an intraocular pressure (IOP); (A2) inputting geometric parameters and material parameters of multi-layer of cornea; (A3) constructing a first corneal numerical model, wherein, using a yield strength of each of the multi-layer as a standard, defining dangerous area if it exceeds the yield strength, defining warning area if it is 60˜100% of the yield strength, and defining safe area if less than 60% of the yield strength; (A4) constructing a second corneal numerical model having at least one cutting path character; (A5-1) inputting a normal IOP value into the second corneal numerical model for simulating, and comparing to the first corneal numerical model, configured to evaluate whether the dangerous area exceeds 5% of whole area of the cornea, or whether the warning area exceeds 20% of whole area of eye (indicated by a first default value); (A5-2) inputting an abnormal IOP value into the second corneal numerical model for simulating, and comparing to the first corneal numerical model, configured to evaluate whether the dangerous area exceeds 10% of whole area of the cornea, or whether the warning area exceeds 50% of whole area of eye (indicated by a second default value); (A5-3) inputting a rubbing-eye IOP value and an external force of a tangent direction or a torque of the cornea into the second corneal numerical model for simulating, and comparing to the first corneal numerical model, configured to evaluate whether the dangerous area exceeds 20% of whole area of the cornea (indicated by a third default value), or whether the warning area exceeds 60% of whole area of eye; (A5-4) inputting a rubbing-eye IOP value and an external force of a tangent direction or a torque of the cornea into the second corneal numerical model for simulating, and comparing to the first corneal numerical model, configured to evaluate whether a stress in any area of each of the multi-layer exceeds 5 times the normal IOP value; (A5-5) inputting a normal IOP value into the second corneal numerical model for simulating, and comparing to the first corneal numerical model, configured to evaluate whether an arc stress in any area of each of the multi-layer exceeds 15%. Wherein the geometrical parameters and material parameters are extracted out during measuring the intraocular pressure.

It is noted that, in step (A5-1)˜step (A5-5) have no sequence, such steps may be assessed individually or in combination, there is not particular limitation.

If the evaluation results of step (A5-1)˜step (A5-5) are negative, proceeding step (A6).

In this embodiment, if the evaluation results of step (A5-1)˜step (A5-5) are positive, proceeding step (A4-1) and performing step (A4) again.

In practical applications, step (A6-1) situational simulation after step (A6-2) establishing a postoperative situation simulation and step (A6-2) establishing a postoperative notice. Specifically, they can be subdivided into step (A7-1) constructing a third corneal numerical model according to a physical appearance of the cornea in postoperative, the model is accordance with a size and a thickness after cutting a physical cornea. In actual situation, the step can be performed one week after surgery. The proceeding step (A7-2) inputting an abnormal IOP value into the third corneal numerical model for simulating to identify a potential high stress area; (A7-3) inputting a rubbing-eye IOP value and an external force of a tangent direction or a torque of the cornea into the second corneal numerical model for simulating to identify a potential high stress area.

In addition, according to the postoperative risk evaluation to proceed step (A8) writing a postoperative patient safety recommendation, the recommendation comprises limitations of life movements, sports and environment.

Similarly, the evaluation method of this embodiment may be built in the evaluation system 3. Using the software and hardware manner to evaluate the stress difference before and after surgery systematically.

Compared to the prior art, the present invention can evaluate difference before and after surgery through a mechanical model and provide a proposed surgical way. It can provide a safer and more accurate evaluation way.

What is claimed is:

1. A corneal surgery risk evaluation method, comprising following steps:
(S1) measuring an intraocular pressure (IOP);
(S2) inputting geometric parameters and material parameters of multi-layer of cornea;
(S3) constructing a first corneal numerical model;
(S4) constructing a second corneal numerical model with at least one cutting path character;
(S5) evaluating whether the cutting path character should be re-constructed or not, wherein the geometrical parameters and material parameters are extracted out during measuring the intraocular pressure.

2. The method as claimed in claim 1, further comprising:
(S6) if the evaluating result is negative, proceeding a postoperative risk evaluation.

3. The method as claimed in claim 1, if the evaluating result of the step (S5) is positive, proceeding step (S4-1) re-constructing the at least one cutting path character, and executing the step (S4) again.

4. The method as claimed in claim 2, further comprising:
(S7) constructing a third corneal numerical model according to an appearance of the cornea in postoperative; and
(S8) establishing a postoperative patient safety recommendation according to the postoperative risk evaluation.

5. The method as claimed in claim 1, wherein the multi-layer at least comprises Bowman's Membrane, Stroma and Descemet's Membrane.

6. The method as claimed in claim 5, wherein the geometric parameters at least comprises curvature and thickness distribution of each of the multi-layer.

7. The method as claimed in claim 5, wherein the material parameters at least comprise Young's modulus, Poisson ratio and yield strength and breaking strength of each of the multi-layer.

8. The method as claimed in claim 4, wherein the postoperative risk evaluation comprises a postoperative situation simulation, the postoperative situation simulation at least comprises regulating IOP value, glare evaluation, and corneal cracking potential.

9. The method as claimed in claim 4, wherein the postoperative risk evaluation and the postoperative patient safety recommendation comprise a postoperative notice, the postoperative notice at least includes calculating a largest acceleration limitation, a largest shearing force limitation, and a largest pressure limitation which the cornea can sustain.

10. The method as claimed in claim 1, wherein the at least one cutting path at least comprises a cutting range, a cutting pattern, a cutting length, and a cutting depth.

11. The method as claimed in claim 1, wherein the step (S5) is based on yield strength of each of the multi-layer.

12. A corneal surgery risk evaluation system, comprising:
a tonometer for providing an external force to cornea and measuring an intraocular pressure;
a camera for measuring a deformation behavior of the cornea; and
a processor connected to the tonometer and the camera, the processor comprises the method as claimed in claim 1.

13. A corneal surgery risk evaluation method, comprising following steps:
(A1) measuring an intraocular pressure (IOP);
(A2) inputting geometric parameters and material parameters of multi-layer of cornea;
(A3) constructing a first corneal numerical model, wherein, using a yield strength of each of the multi-layer as a standard,
defining dangerous area if it exceeds the yield strength,
defining warning area if it is 60˜100% of the yield strength, and
defining safe area if less than 60% of the yield strength;
(A4) constructing a second corneal numerical model having at least one cutting path character;
(A5-1) inputting a normal IOP value into the second corneal numerical model for simulating, and comparing to the first corneal numerical model, configured to evaluate whether the dangerous area exceeds 5% of whole area of the cornea, or whether the warning area exceeds 20% of whole area of eye;
(A5-2) inputting an abnormal IOP value into the second corneal numerical model for simulating, and comparing to the first corneal numerical model, configured to evaluate whether the dangerous area exceeds 10% of whole area of the cornea, or whether the warning area exceeds 50% of whole area of eye;
(A5-3) inputting a rubbing-eye IOP value and an external force of a tangent direction or a torque of the cornea into the second corneal numerical model for simulating, and comparing to the first corneal numerical model, configured to evaluate whether the dangerous area exceeds 20% of whole area of the cornea, or whether the warning area exceeds 60% of whole area of eye;
(A5-4) inputting a rubbing-eye IOP value and an external force of a tangent direction or a torque of the cornea into the second corneal numerical model for simulating, and comparing to the first corneal numerical model, configured to evaluate whether a stress in any area of each of the multi-layer exceeds 5 times the normal IOP value;
(A5-5) inputting a normal IOP value into the second corneal numerical model for simulating, and comparing to the first corneal numerical model, configured to evaluate whether an arc stress in any area of each of the multi-layer exceeds 15%,
wherein the geometrical parameters and material parameters are extracted out during measuring the intraocular pressure.

14. The method as claimed in claim 13, further comprising:
(A6) if the evaluating result of the step (A5-1)˜step (A5-5) is negative, proceeding a postoperative risk evaluation.

15. The method as claimed in claim 14, wherein the postoperative risk evaluation comprising following step:
(A6-1) establishing a postoperative situation simulation, at least including regulating IOP value, glare evaluation, and corneal cracking potential.

16. The method as claimed in claim 13, further comprising:
(A6-2) establishing a postoperative notice, at least including calculating a largest acceleration limitation, a largest shearing force limitation, and a largest pressure limitation which the cornea can sustain.

17. The method as claimed in claim 13, if the evaluating result of the step (A5-1)˜step (A5-5) is positive, proceeding step (A4-1) re-constructing the at least one cutting path character, and executing the step (A4) again.

18. The method as claimed in claim 15, further comprising:
(A7-1) constructing a third corneal numerical model according to a physical appearance of the cornea in postoperative, the model is accordance with a size and a thickness after cutting a physical cornea;
(A7-2) inputting an abnormal IOP value into the third corneal numerical model for simulating to identify a potential high stress area;
(A7-3) inputting a rubbing-eye IOP value and an external force of a tangent direction or a torque of the cornea into the second corneal numerical model for simulating to identify a potential high stress area; and
(A8) writing a postoperative patient safety recommendation, the recommendation comprises limitations of life movements, sports and environment.

19. The method as claimed in claim 16, further comprising:
(A7-1) constructing a third corneal numerical model according to a physical appearance of the cornea in postoperative, the model is accordance with a size and a thickness after cutting a physical cornea;
(A7-2) inputting an abnormal IOP value into the third corneal numerical model for simulating to identify a potential high stress area;
(A7-3) inputting a rubbing-eye IOP value and an external force of a tangent direction or a torque of the cornea into the second corneal numerical model for simulating to identify a potential high stress area; and
(A8) writing a postoperative patient safety recommendation, the recommendation comprises limitations of life movements, sports and environment.
